(19) United States
(12) Patent Application Publication
(10) Pub. No.: US 2012/0224995 A1
(43) Pub. Date: Sep. 6, 2012

(54) FRAGRANCE WARMER WITH WATER-BASED FRAGRANCE GEL

(76) Inventor: David W. McMinn, Marshall, TX (US)

(21) Appl. No.: 13/039,983

(22) Filed: Mar. 3, 2011

(51) Int. Cl. A61L 9/00; A61Q 13/00; A61K 8/34; C11D 3/50

(52) U.S. Cl. 422/4; 422/125; 512/4; 512/2

(57) ABSTRACT

An improved fragrance warmer, water-based fragrance gel, and related methods of use are disclosed. In one aspect, the Fragrance warmer has at least high and low heat settings selected for use with a water-based fragrance gel. In other aspects, a water based fragrance gel is provided that can, in certain embodiments, be made with de-ionized water to achieve a viscosity approximately residing a range of about 19,000 centipoises to about 30,000 centipoises. In further aspects, a method of using the water-based gel includes observing that the gel on a warmer tray has reduced to a residue, and cleaning the warmer tray by hand washing it with soap and water, or by running it through a dishwasher.

### TECHNICAL FIELD

The present disclosure relates generally to scent dispensers, fragrance media, and related methods of use, and even more particularly to fragrance warmers, fragrance gels, and related methods of use.

### BACKGROUND

Today's scent dispensing systems take a variety of forms, including incense, scented candles, electric air fresheners, oil warmers/burners, and reed diffusers. Although scented candles are very popular, a related option that does not entail an open flame is a fragrance warmer, of which a candle warmer is a common variety. A candle warmer is an electric warmer that melts a candle or scented wax to release its scent. Some candle warmers are intended to be used with jar candles or candles in cups, but not with taper candles or candles without containers large enough to accommodate all of the melted wax. Other candle warmers have a built-in bowl in which a candle can be placed.

The advantages of using a candle warmer include the absence of open flame and the soot that often results from burning wax. Many warmers are designed to be used with “wickless” candles, which are blocks or lumps of scented candle wax with no wick. One disadvantage of a candle warmer is difficulty for users to judge the amount remaining fragrance. This difficulty arises because the wax offers no visible indication as to the amount of fragrance remaining in the wax. A hot pool of wax forms that eventually stops throwing fragrance, but users remain unaware of the need to replace the wax melt material until the fragrance dissipates from the area. Another disadvantage of wax melts is difficult cleanup. Because wax stiffens when cooled, it can adhere to many surfaces and prove difficult to remove. Thus, the user is required clean the warmer tray by pouring hot wax out of the warmer tray into a waste receptacle with the risk of spilling the hot wax.

Other fragrance warmers have used oil based solutions or gels instead of wax melt. Such warmers have an advantage of a strong fragrance throw, allowing a single warmer to provide more intense scent to an area, or to provide a scent to a larger area. Another advantage of such warmers is the visible reduction of the fragrance medium during use that provides users with clear indication of a need to replace the fragrance medium. However, oil based media, once utilized, can be difficult to clean from a warmer surface. Additionally, oil based media are prone to splatter as they reduce in volume during use, thus covering surrounding surfaces with an unpleasant oily residue that can be difficult to clean, and that can cause permanent oil stains to fabrics and other absorbent surfaces.

### SUMMARY

A fragrance warmer, water-based fragrance gel, and related methods of use are disclosed. In one aspect, the Fragrance warmer has at least high and low heat settings selected for use with a water-based fragrance gel. In other aspects, a water based fragrance gel is provided that can, in certain embodiments, be made with de-ionized water to achieve a viscosity approximately residing a range of about 19,000 centipoises to about 30,000 centipoises. In further aspects, a method of using the water-based gel includes observing that the gel on a warmer tray has reduced to a residue, and cleaning the warmer tray by hand washing it with soap and water, or by running it through a dishwasher.

### BRIEF DESCRIPTION OF THE DRAWINGS

FIG. 1 is a rising view of a first embodiment of a fragrance warmer in accordance with the teachings of the present disclosure.

FIG. 2 is a cross-sectional structural diagram of the first embodiment of the fragrance warmer of FIG. 1 in accordance with the teachings of the present disclosure.

FIG. 3 is an overhead view of the first embodiment of the fragrance warmer of FIG. 1 in accordance with the teachings of the present disclosure.

FIG. 4 is a descending view of the first embodiment of the fragrance warmer of FIG. 1 in accordance with the teachings of the present disclosure.

FIG. 5 is an overhead view of a lamp element of the first embodiment of the fragrance warmer of FIG. 3 in accordance with the teachings of the present disclosure.

FIG. 6 is an overhead view of an electrical socket of the lamp element of FIG. 5 in accordance with the teachings of the present disclosure.

FIG. 7 is a rising view of a second embodiment of a fragrance warmer in accordance with the teachings of the present disclosure.

FIG. 8 is a cross-sectional structural diagram of the second embodiment of the fragrance warmer of FIG. 7 in accordance with the teachings of the present disclosure.

FIG. 9 is a rising view of a third embodiment of a fragrance warmer in accordance with the teachings of the present disclosure.

FIG. 10 is a cross-sectional structural diagram of the third embodiment of the fragrance warmer of FIG. 9 in accordance with the teachings of the present disclosure.

FIG. 11 is a rising view of a fourth embodiment of a fragrance warmer in accordance with the teachings of the present disclosure.

FIG. 12 is a cross-sectional structural diagram of the fourth embodiment of the fragrance warmer of FIG. 11 in accordance with the teachings of the present disclosure.

FIG. 13 is a rising view of a fifth embodiment of a fragrance warmer in accordance with the teachings of the present disclosure.

FIG. 14 is a cross-sectional structural diagram of the fifth embodiment of the fragrance warmer of FIG. 13 in accordance with the teachings of the present disclosure.

FIG. 15 is a rising view of a sixth embodiment of a fragrance warmer in accordance with the teachings of the present disclosure.

FIG. 16 is a cross-sectional structural diagram of the sixth embodiment of the fragrance warmer of FIG. 15 in accordance with the teachings of the present disclosure.

FIG. 17 is a rising view of a seventh embodiment of a fragrance warmer in accordance with the teachings of the present disclosure.

FIG. 18 is a cross-sectional structural diagram of the seventh embodiment of the fragrance warmer of FIG. 17 in accordance with the teachings of the present disclosure.

FIG. 19 is a rising view of an eighth embodiment of a fragrance warmer in accordance with the teachings of the present disclosure.

FIG. 20 is a cross-sectional structural diagram of the eighth embodiment of the fragrance warmer of FIG. 19 in accordance with the teachings of the present disclosure.

FIG. 21 is a rising view of a ninth embodiment of a fragrance warmer in accordance with the teachings of the present disclosure.

FIG. 22 is a cross-sectional structural diagram of the ninth embodiment of the fragrance warmer of FIG. 21 in accordance with the teachings of the present disclosure.

FIG. 23 is a rising view of a tenth embodiment of a fragrance warmer in accordance with the teachings of the present disclosure.

FIG. 24 is a cross-sectional structural diagram of the tenth embodiment of the fragrance warmer of FIG. 23 in accordance with the teachings of the present disclosure.

FIG. 25 is a rising view of an eleventh embodiment of a fragrance warmer in accordance with the teachings of the present disclosure.

FIG. 26 is a cross-sectional structural diagram of the eleventh embodiment of the fragrance warmer of FIG. 25 in accordance with the teachings of the present disclosure.

FIG. 27 is a rising view of a water-based fragrance gel placed on a warming surface of a fragrance warmer in accordance with the teachings of the present disclosure.

FIG. 28 is a rising view of a container for storing the water-based fragrance gel and dispensing the water-based fragrance gel onto the warming surface of the fragrance warmer in accordance with the teachings of the present disclosure.

FIG. 29 is a flow diagram illustrating a method of making and packaging the water-based fragrance gel in accordance with the teachings of the present disclosure.

FIG. 30 is a flow diagram illustrating a method of using the fragrance warmer and water-based fragrance gel in accordance with the teachings of the present disclosure.

FIG. 31 is a rising view of the container for storing and dispensing the water-based fragrance gel according to measurements marked on a label affixed to an at least partially light transmissive surface of the container in accordance with the teachings of the present disclosure.

FIG. 32 is a rising view illustrating dispensing of the water-based fragrance gel onto the warming surface of the fragrance warmer in accordance with the teachings of the present disclosure.

FIG. 33 is a rising view illustrating a single measured amount of the fragrance gel dispensed on the warming surface of the fragrance warmer in accordance with the teachings of the present disclosure.

FIG. 34 is an overhead view illustrating a residue remaining on the warming surface of the fragrance warmer upon complete reduction of the water-based fragrance gel in accordance with the teachings of the present disclosure.

### DETAILED DESCRIPTION

In the following description, like elements are marked throughout the specification and drawings with the same reference numerals, respectively. The drawing figures are not to scale and certain elements are shown in generalized or schematic form in the interest of clarity and conciseness. Dimensions illustrated are merely exemplary, and can be modified as desired. It should be understood that the embodiments of the disclosure herein described are merely illustrative of the application of the principles of the disclosure.

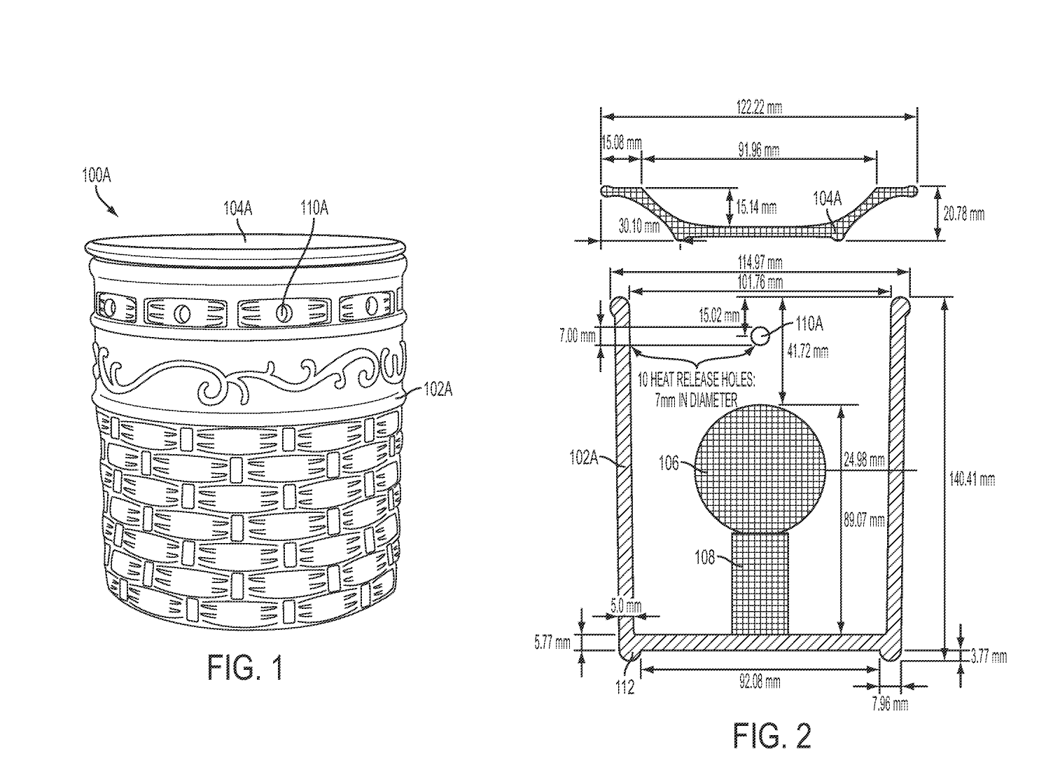

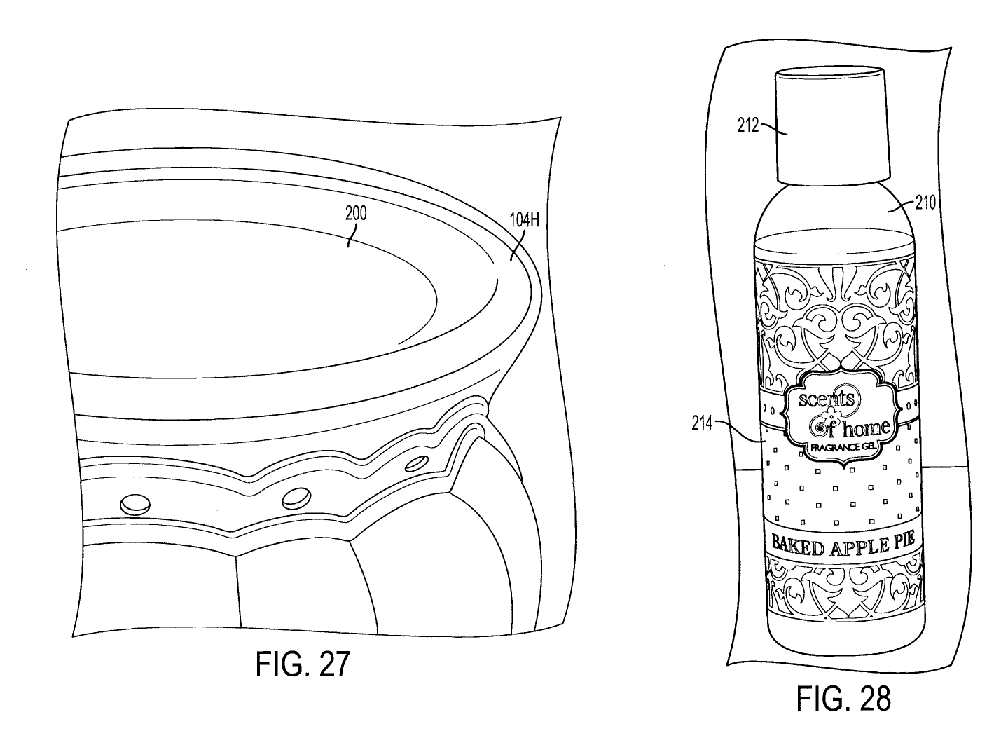

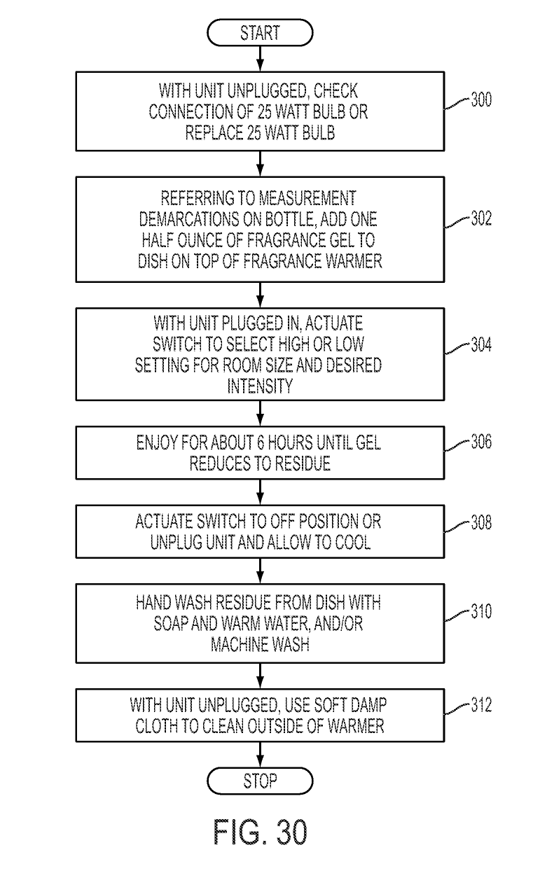

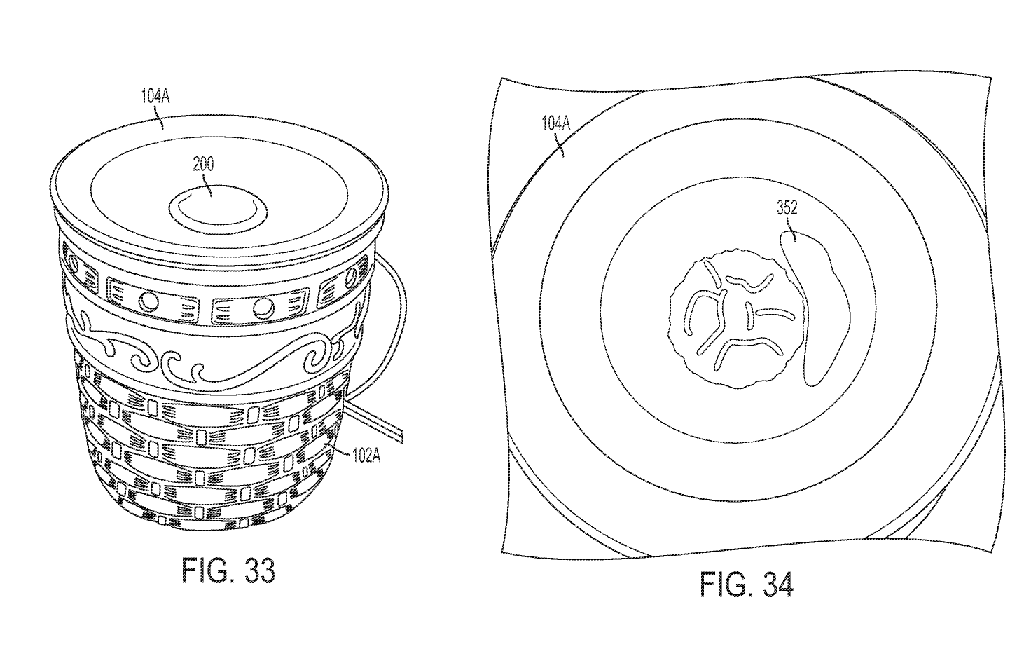

The present disclosure provides an improved fragrance warmer, an improved, water-based fragrance gel, and a related method of use. Embodiments of the improved fragrance warmer are described in detail below with reference to FIGS. 1-26. Embodiments of the improved, water-based fragrance gel are described in detail below with reference to FIGS. 27-29. Embodiments of the related method of use are described below with reference to FIGS. 30-34.

Turning to FIGS. 1 and 2 and referring generally thereto, a first embodiment of a fragrance warmer 100A can be implemented as a fragrance lamp having a warmer body 102A configured to support a warmer tray 104A, both of which can be formed of specifically blended stoneware and covered with glaze. For example, the fragrance warmer 100A can be composed of sculpted stoneware and covered with a white glaze in a manner causing it to resemble a cylindrical wicker basket. The stoneware can exhibited a heat tolerance of 1260 degrees centigrade, water absorption greater than 1.5%, and shrinkage greater than 12%.

The warmer body 102A can be, for example, 140.41 mm high and 114.97 mm in diameter, while the warmer tray 104A can be 20.78 mm high, 122.22 mm in external diameter, 91.96 mm in internal diameter, and 15.14 mm deep. The stoneware can have a thickness of about 5 mm to 6 mm. The warmer body 102A can have an internal diameter of 114.97 mm, and house a lamp element including, for example, a 25 watt, incandescent, AC, electric light bulb 106 (e.g., 120V 25 Watt candelabra type) that physically interfaces in electrical communication with a electrical socket 108 (e.g., 45 E2 lampholder 125V 75 Watt Gold) that can be composed of porcelain and mounted in a base of the warmer body 102A. Other or different dimensions can additionally or alternatively be employed.

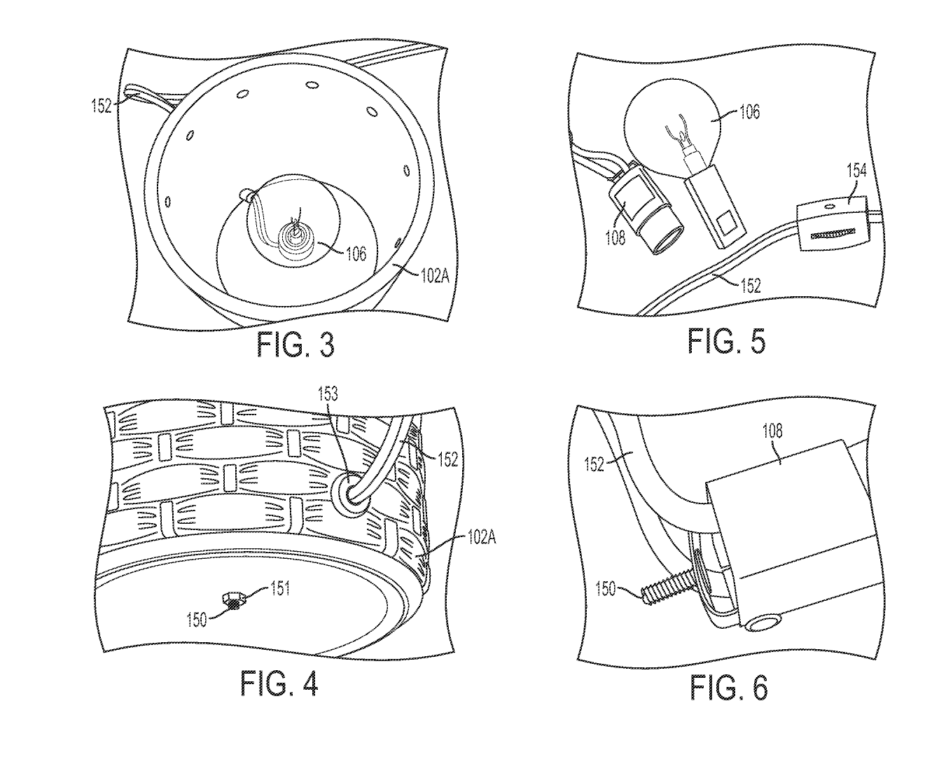

A number of heat release holes 110A (e.g., ten) can be formed in an upper area of the warmer body 102A, and evenly distributed in a line circumscribing the cylindrical warmer body. These heat release holes 110A can have a number and diameter (e.g., 7 mm) selected in view of interior volume of warmer body 102A and operational settings and characteristics of light bulb 106 to achieve target heat levels (e.g., a variance of 135 degrees Fahrenheit to 160 degrees Fahrenheit, etc.) for the warmer surface of warmer tray 104A. Feet 112 formed in the base of the warmer body 102A can permit a clearance for a lamp element mounting apparatus (e.g. 7 mm nut and bolt) as further described below with reference to FIGS. 3-6.

Turning now to FIGS. 3-6 and referring generally thereto, the lamp element can mount vertically to the base warmer body 102A by passing a metal, threaded support member 150 (e.g., 7 mm bolt) extending from a lower portion of the electrical socket 108 through a through hole formed in the base of the lamp body 102A. A nut 151 or other suitable mechanism can be fitted onto the threaded member from beneath the base to affix the electrical socket 108 in an upright position that situates light bulb 106 in an approximate center of the interior of the warmer body 102A. An electrical cable 152 (e.g., (UL) E1577 SPT-2), that can also extend from the base of the electrical socket 108, can similarly pass through another through hole formed in a lower portion of the warmer body 102A and provided with a mounting bracket 153 that can be made, for example, of plastic. Electrical cable 152 can have a switch 154 (e.g., single pole throw 2 way) that can be actuated by a user to turn the lamp element on and off, and to select one of multiple heat settings. For example, a low heat setting (e.g., 135 degrees Fahrenheit, etc.) can be selected by rotating the switch from the off position to a first position. Then, a high heat setting (e.g., 160 degrees Fahrenheit, etc.) can be selected by rotating the switch 154 to the next position. Thereafter, the unit can be returned to the off position by rotating the switch 154 once more.

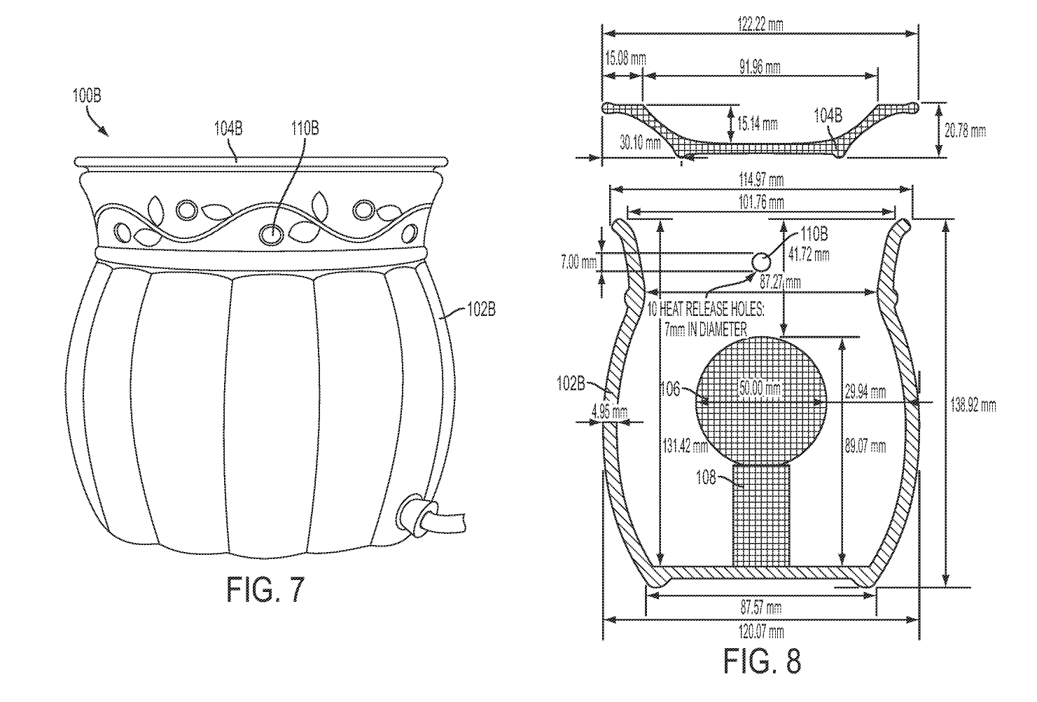

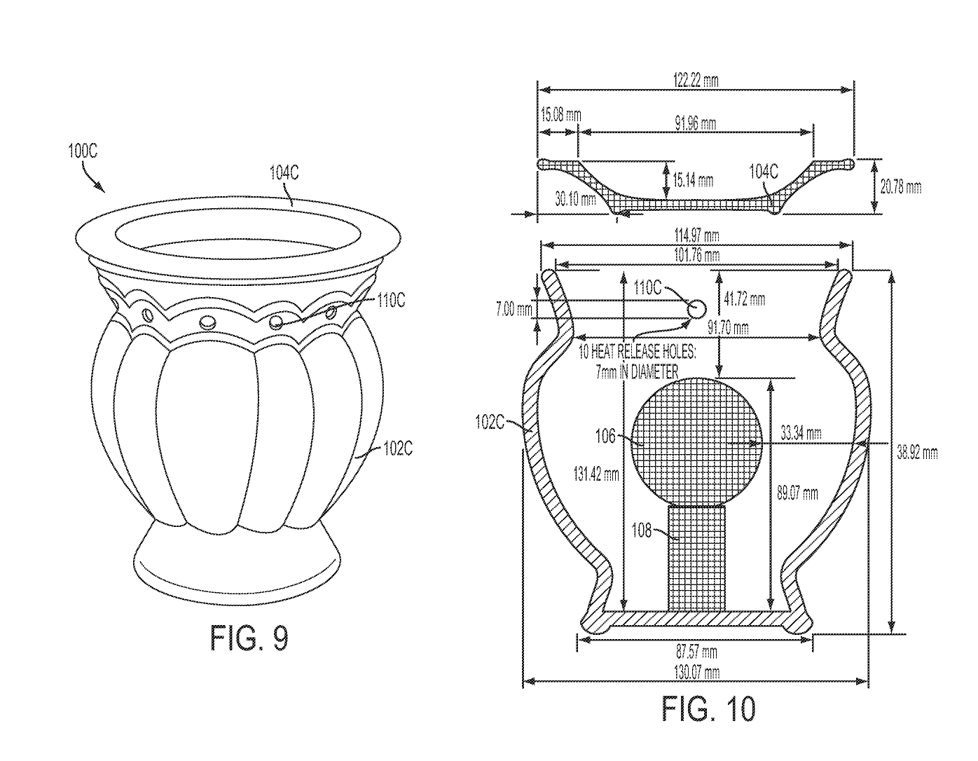

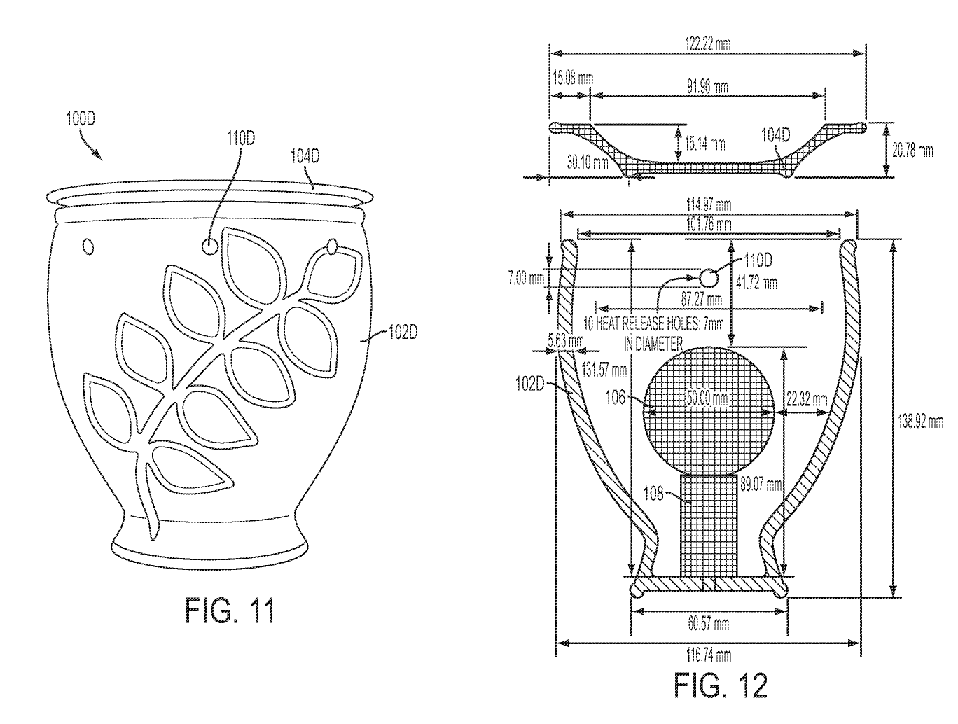

Turning now to FIGS. 7-18 and referring generally thereto, other embodiments of the fragrance warmer 100B-100G can have composition, dimensions, elements (e.g., bulb 106 and socket 108), features (e.g., holes 110B-110G), and functionality that are identical or very similar those of the first embodiment, but achieve a wide variety in appearance by differences in sculpture of the warmer bodies 102B-102G and/or trays 104B-104G and the glazing selection. For example, referring to FIGS. 7 and 8, a second embodiment of the fragrance warmer 100B can have composition, dimensions, elements, features, and functionality that are identical or very similar to those of the first embodiment, while being sculpted to have an appearance of a fluted vase, and coated in a berry under glaze having a clear over glaze. The heat release holes 1108 can be arranged in two lines with a staggered appearance without significantly altering the functionality of the warmer 1008. Similarly, referring to FIGS. 9 and 10, a third embodiment of the fragrance warmer 100C can have composition, dimensions, elements, features, and functionality that are identical or very similar to those of the first embodiment, while being sculpted to have an appearance of venetian pottery embossed with a fluted pattern, and coated in a yellow and brown under glaze having a clear over glaze. Additionally, referring to FIGS. 11 and 12, a fourth embodiment of the fragrance warmer 100D can have composition, dimensions, elements, features, and functionality that are identical or very similar to those of the first embodiment, while being sculpted to have an appearance of pottery embossed with an image of a leafy growth, and having a light brown/titanium reactive over glaze.

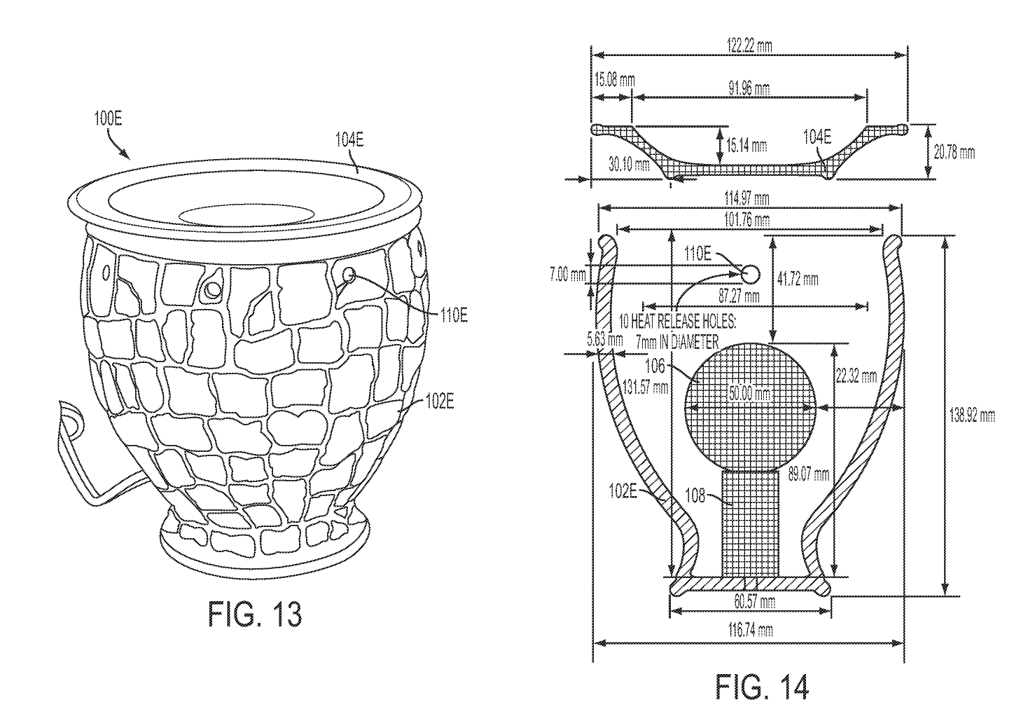

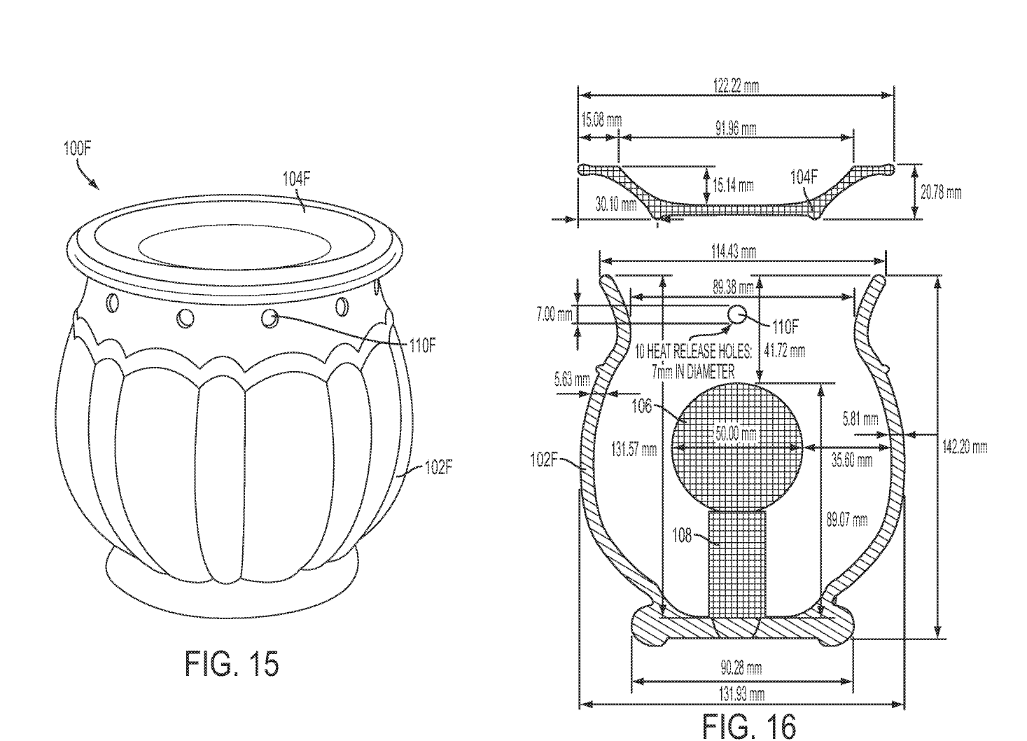

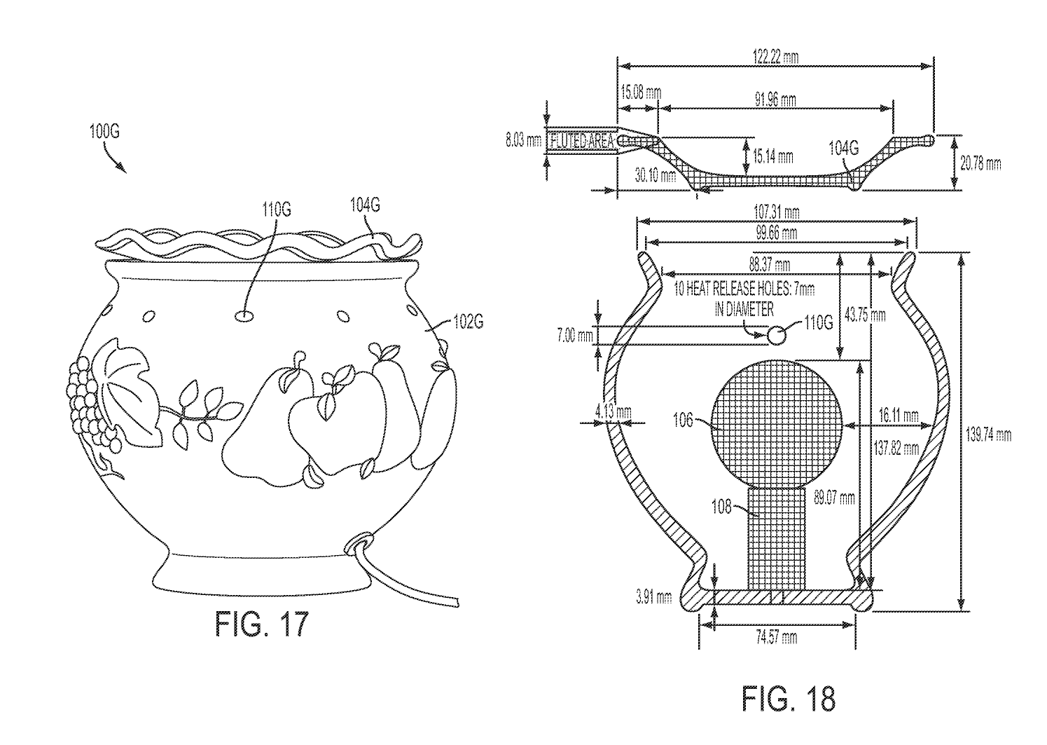

Additional variety in appearance of the warmer can be achieved by varying the sculpture of the warmer body and the glazing selection without adjusting the overall internal warmer volume or number or size of heat release holes. For example, referring to FIGS. 13 and 14, a fifth embodiment of the fragrance warmer 100E can have composition, dimensions, elements, features, and functionality that are identical or very similar to those of the first embodiment, while being sculpted to have an appearance of pottery embossed with a pattern of crocodile scales, and having a black/brown reactive over glaze. Additionally, referring to FIGS. 15 and 16, a sixth embodiment of the fragrance warmer 100F can have composition, dimensions, elements, features, and functionality that are identical or very similar to those of the first embodiment, while being sculpted to have an appearance of pottery embossed with a fluted pattern, and having a feldspathic vanadium glaze. Further, referring to FIGS. 17 and 18, a seventh embodiment of the fragrance warmer 100G can have composition, dimensions, elements, features, and functionality that are identical or very similar to those of the first embodiment, while being sculpted to have an appearance of pottery embossed with a fluted pattern in both the warmer body 102G and the warmer tray 102G, and having a white glaze and hand painted florals and fruits. Thus, a wide variety can be achieved in the shape and appearance of the warmer without substantially modifying the overall internal volume and functional features of the warmer.

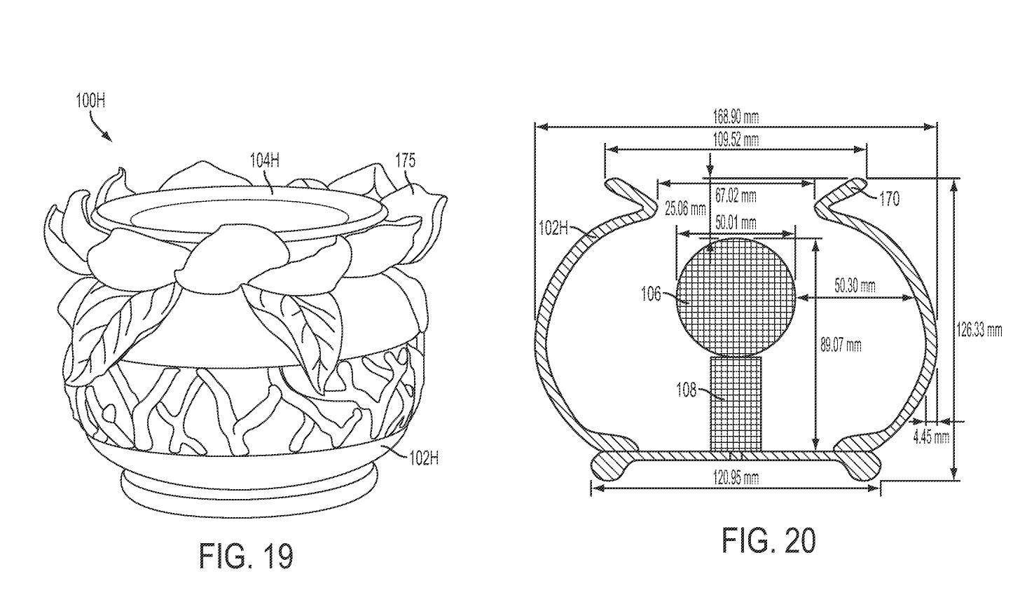

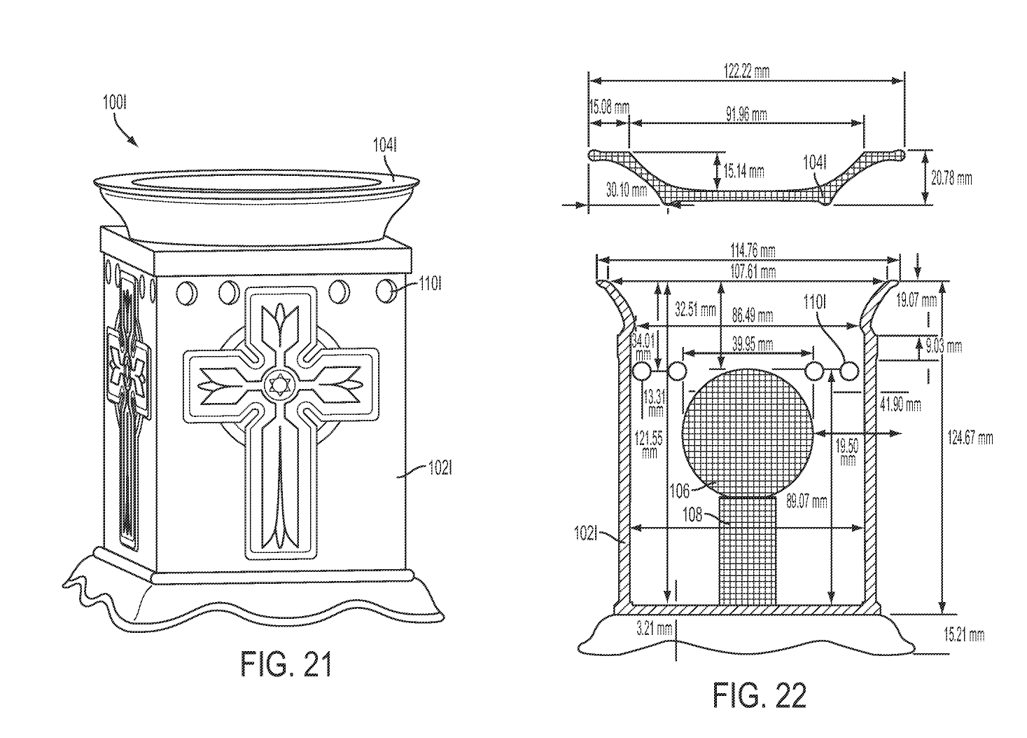

Turning now to FIGS. 19-26 and referring generally thereto, additional embodiments of the fragrance warmer 100H-100K can achieve an even greater variety in appearance by implementing modified dimensions and modified heat release features while still achieving the target heat settings with the 25 watt bulb 106 mounted in socket 108. For example, referring to FIGS. 19 and 20, an eighth embodiment of the fragrance warmer 100H can have a warmer body 102H that is shorter (e.g., 126.33 mm) but wider to provide a larger volume. Formed of a cast polyester resin coated with polyurethane paint, it can have an upper lip 170 of the warmer body sculpted in a formation (e.g., magnolia blossom petals 175) that supports a clear glass warmer tray 104H above heat release vents (e.g., spaces between the petals 175) formed in the upper lip 170. This clear glass warmer tray 104H can have an etched and/or rough surface to diffuse light, thus permitting the warmer 100H to function as a night light. This clear glass warmer tray 104H can have a standard size that allows it to fit other embodiments of the warmer to allow those warmers to also function as night lights. In some embodiments, the clear glass warmer tray 104H can be purchased separately and substituted for a stoneware warmer tray or a porcelain warmer tray. Additionally, referring to FIGS. 21 and 22, a ninth embodiment of the fragrance warmer 100I can have a shorter warmer body 102I (e.g., 124.67 mm) that is generally cubical (e.g., 126.33 mm) in shape, thus having significantly less volume. This embodiment of the fragrance warmer 100I and its warmer tray 104I can be composed of unglazed bone china porcelain that can be partially light transmissive to provide a pleasing glow when lit. It can also have a greater number (e.g. sixteen) of heat release holes 110I arranged linearly in pairs near the upper corners of the cubically shaped fragrance warmer 100I.

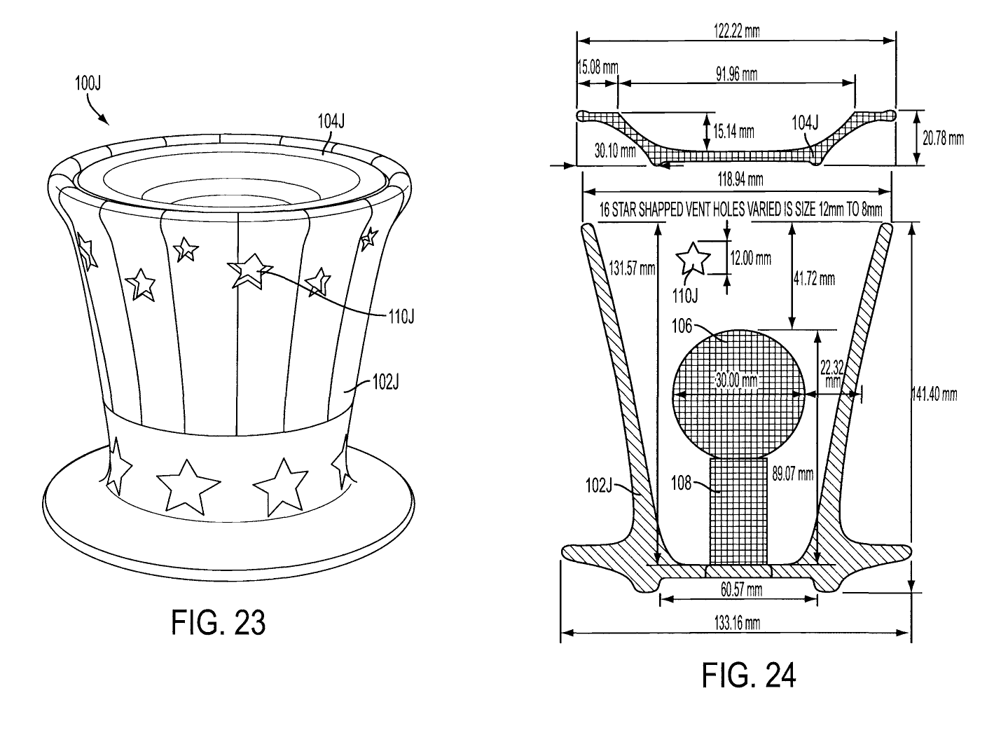

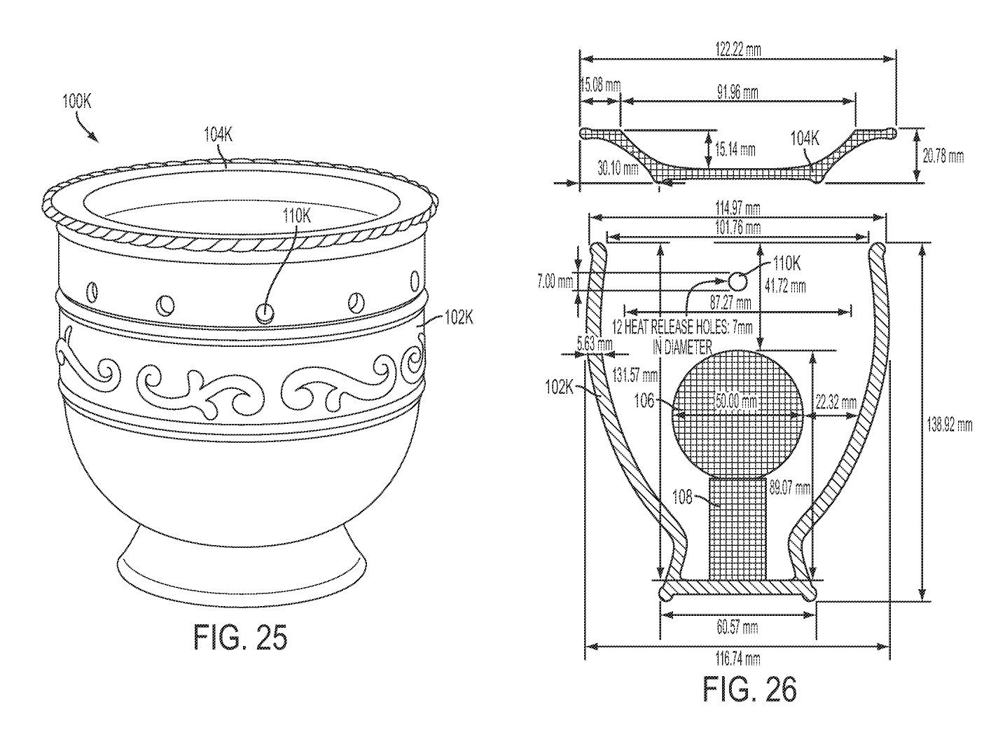

Other embodiments of the fragrance warmer can also achieve a greater variety in appearance by implementing modified dimensions and modified heat release features while still achieving the target heat settings with the 25 watt bulb. For example, referring to FIGS. 23 and 24, a tenth embodiment of the fragrance warmer 100J can be formed of dolomite with under glaze, and provided with red and blue under paint and clear over glaze to have the appearance of a top hat. Somewhat conically shaped, the warmer body 102J can have a smaller diameter (e.g., 60.57 mm) at the bottom of the fragrance warmer 100J, resulting in a decreased internal volume. To compensate, a greater number of polygonal heat release holes 110J (e.g., 16 stars) can be provided of varying sizes (e.g., 8 mm to 12 mm) and staggered in two circumferential lines near the top of the fragrance warmer, below the warmer tray 104J. Also, referring to FIGS. 25 and 26, an eleventh embodiment of the fragrance warmer 100K can be shaped, for example, as an urn having an under glaze and yellow and orange over glaze, with a raised scroll pattern sculpted on the body 102K and rope sculpted about a rim of the warmer tray 104K. Being of slightly lesser height (e.g., 138.92 mm) and significantly decreased lower diameter (e.g., 60.57 mm) it can have a substantially decreased volume. To compensate, a greater number (e.g., twelve) of heat release holes 110K can be provided about the top portion of the warmer body 102K. Thus, various embodiments of the fragrance warmer can have significant variations in internal warmer volume by adjusting the number, size, and shape of heat release holes, and or by using vents formed in an upper lip of the warmer body. Thus, it can be appreciated that the dimensions and features of the fragrance warmer can be varied while still achieving the target heat settings, and that further variations are possible. Moreover, it should be readily understood that alternative or additional heat sources can be utilized that provide more or less thermal power than the 25 Watt bulb, and that adjustments can be made to the warmer dimensions and heat release features to accommodate a great deal of variety.

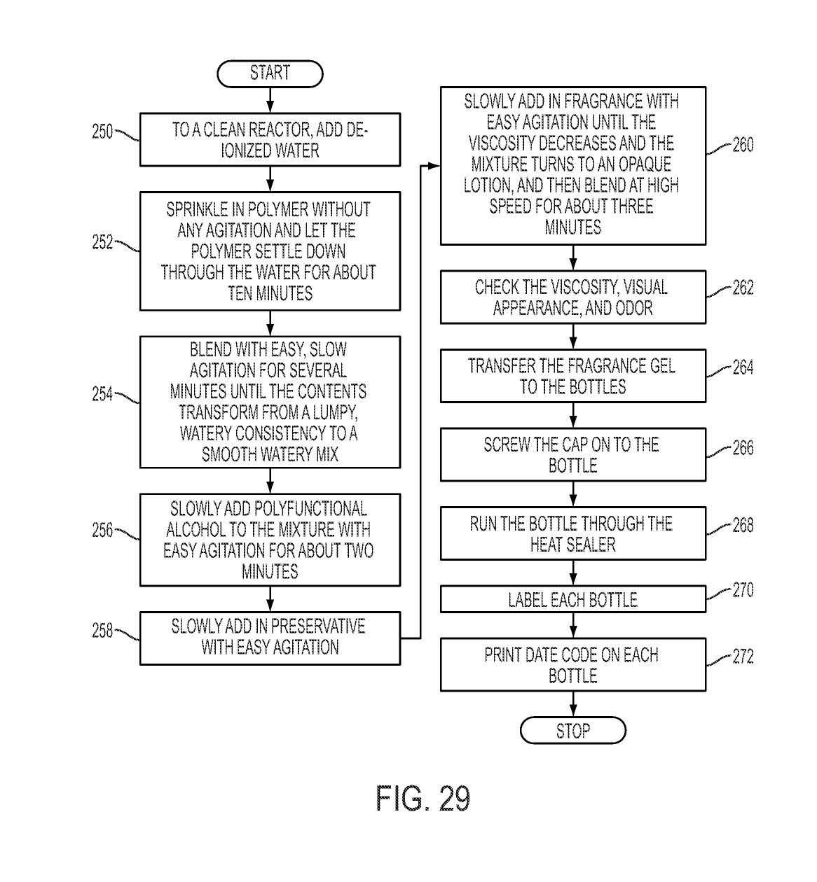

Turning now to FIGS. 27-29, a water-based fragrance gel 200 is disclosed in detail. Referring to FIG. 27, an amount of the water-based fragrance gel 200 placed in clear glass warmer tray 104H atop a fragrance warmer can have a viscosity that can allow it to spread out across a portion of a surface of the tray 202. The viscosity of the gel can also ensures that a small amount of the gel 200 does not necessarily spread across the entire bottom surface of the tray and become a thin layer, as would normally occur with similar amounts of a liquid or heated oil product. Thus, the viscosity can permit various amounts of the gel 200 to spread out across the surface of the tray 202 to a degree that it neither piles up, nor spreads out to a thin layer like a liquid or oil. Unlike oil, the water-based gel 200 does not liquefy, spread, pool, or splatter when heated, but foams at edges while reducing to a dry residue. The viscosity that achieves these characteristics is similar to that of many hair gels and/or hand lotions, and it can be achieved in part by making the gel 200 using de-ionized water as further explained below with reference to FIG. 29.

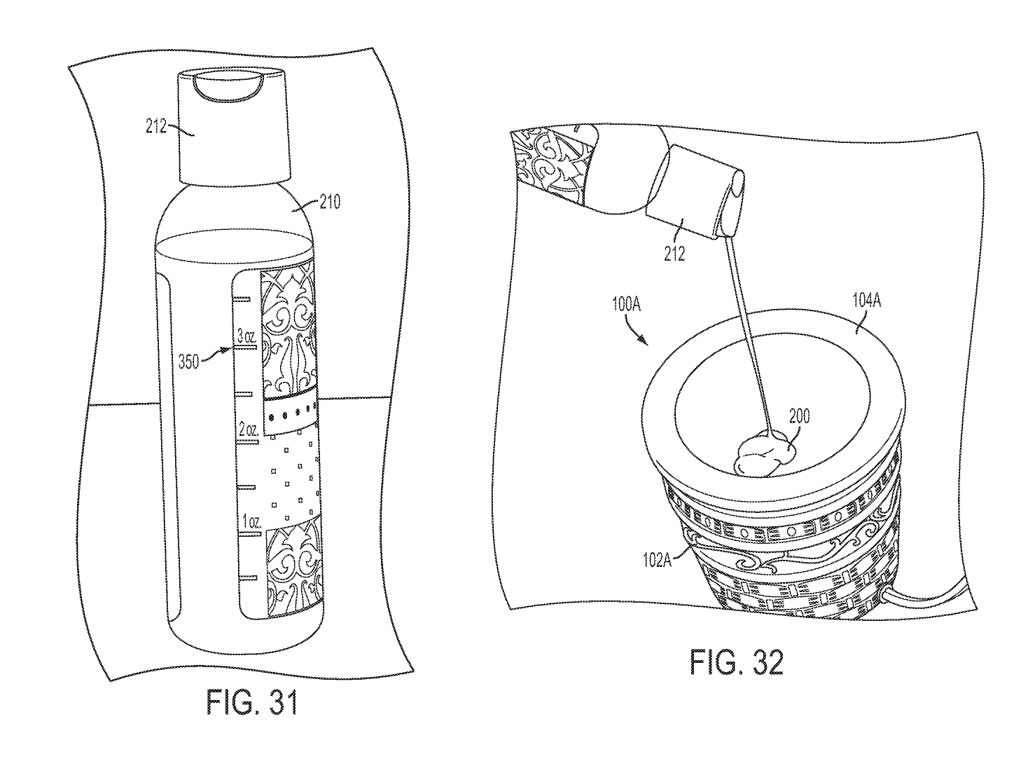

Referring to FIG. 28, a container 210 for storing and dispensing the gel can be made of plastic and sized to contain an amount of the gel (e.g., four ounces) that provides enough gel for multiple uses. This container 210 can have a cap 212 that can screw onto the container and have a feature (e.g., pop up spout) for dispensing the gel. Features of containers for dispensing hair care and/or skin care products (e.g., hair gel, hand lotion, etc.) can be suitable. A paper label 214 affixed to the surface of the container 210 can identify the particular fragrance of the gel and provide instructions to users, and a date stamp on the container 210 can ensure freshness. As discussed in more detail below with reference to FIG. 31, the label can have lines that indicate gel usage amounts in increments (e.g., one-half ounce), and the container 210 can be at least partially light transmissive to permit users to view the amount of gel remaining in the container 210 with reference to the indices. It is envisioned that other embodiments of the container 210 can have indices formed into or printed directly onto the container 210.

Turning now to FIG. 29, various embodiments of the water-based fragrance gel can be made by blending fragrance with water (e.g., de-ionized water), one or more polymers (e.g., Ultrez 21 polymer), one or more polyfunctional alcohols (e.g., triethanolamine), and one or more preservatives (e.g., phenonip preservative). Some example fragrances that can be used in the gel can be a sandalwood vanilla fragrance, a baked apple pie fragrance, an orange vanilla fragrance, a mango cooler fragrance, a gardenia fragrance, and a kiwi papaya fragrance. The sandalwood vanilla fragrance can include scents of white floral touched with raspberry and peach, sandalwood and creamy vanilla. The baked apple pie fragrance can include scents of apples baked in cinnamon, sugar, and vanilla. The orange vanilla fragrance can include scents of juicy orange zest and vanilla. The mango cooler fragrance can include scents of mango, tangerine, lemon, and star fruits with red grape. The gardenia fragrance can include scents of gardenia mingled with rose, jasmine, lime, eucalyptus and wintergreen. The kiwi papaya fragrance can include scents of juicy melon, papaya, and kiwi, with a touch of bananas, peaches, and vanilla beans.

In terms of parts by weight, the water-based fragrance gels, in some embodiments, are generally made using about 10% fragrance and about 0.5% preservative. The amounts of water, polymer, and polyfunctional alcohol can vary in each formulation, but the amounts generally can lie in certain ranges, and the amounts of polymer and polyfunctional alcohol can be substantially equal to one another. For example, as detailed below in Table 1, an amount of de-ionized water can generally be about 89.1% to about 89.2%. Similarly, an amount of Ultrez 21 polymer can be about 0.15% to about 0.20%, and an amount of triethanolamine can be a substantially equal amount of about 0.15% to about 0.20%. Without undue experimentation, one skilled in the art should readily recognize variations in the amounts, as well as alternative and/or additional ingredients in similar or dissimilar amounts, that can have substantially the same function in substantially the same way to achieve substantially similar gels of which the ornamental aspects (i.e., selected fragrances) can be similar or dissimilar, as desired.

TABLE 1

| | | Ultrez | |
|---|---|---|---|
| Fragrance | De-Ionized Water | 21 Polymer | Triethanolamine |
| Sandalwood Vanilla | 89.1% | 0.20% | 0.20% |
| Baked Apple Pie | 89.15% | 0.175% | 0.175% |
| Orange Vanilla | 89.15% | 0.175% | 0.175% |
| Mango Cooler | 89.18% | 0.16% | 0.16% |
| Gardenia | 89.2% | 0.15% | 0.15% |

In some embodiments, a process for blending the aforementioned ingredients can begin at step 250 by adding water to a clean reactor. To achieve the presently preferred viscosity in the final gel product, de-ionized water can be used. In some embodiments, an amount of water that can be added can lie in a range of about 89.1% to about 89.2% by weight. Next, in step 252, the polymer can be sprinkled in without any agitation, and the polymer can be allowed to settle down through the water for about ten minutes. For example, an amount of Ultrez 21 polymer can be added in step 252. In some embodiments, the amount of polymer added in step 252 can lie in a range of about 0.15% to about 0.20% by weight. Then, in step 254, the polymer and water can be blended with easy, slow agitation for several minutes until the contents transform from a lumpy, watery consistency to a smooth, watery mix.

Once the smooth, watery mix has been obtained, the easy agitation can continue in step 256 as a polyfunctional alcohol is added for about two minutes. In some embodiments, the polyfunctional alcohol can be triethanalomene, and the amount of polyfunctional alcohol that is added can be substantially the same amount by weight as the polymer. In other words, the amount of polyfunctional alcohol added in step 256 can lie in a range of about 0.15% to about 0.20% by weight. At this point the mixture can be observably thicker, and it can have cleared to about the consistency of a hair gel. Next, at step 258, a preservative can be added with easy agitation. In some embodiments, the preservative can be phenonip preservative, and the amount of preservative added in step 258 can be about 0.5% by weight. Then, at step 260, the fragrance can be added. In some embodiments, the amount of fragrance added in step 260 can be about 10% by weight. The fragrance can be added with easy agitation until the viscosity decreases and the mixture turns to an opaque lotion, followed by blending at high speed for about three minutes. The resulting mixture is the finished fragrance gel.

Once the gel is finished, the viscosity, visual appearance, and odor can be checked at step 262. To check the viscosity, the gel can be tested, for example, using a CSC Bostwick Consistometer, which is a device that accurately determines sample consistency by measuring the distance a material flows under its own weight during a given time interval. The “Bostwick” is a long trough with 0.5 cm graduations along the bottom. The trough is separated near one end by a spring loaded gate, thus forming a chamber where the sample can be loaded. To perform a test, first a sample is loaded, the then the gate is opened and a timer is started. At a predetermined time, the position of the sample in the trough is recorded. A Consistometer reading that lies in a range of about 5 cm to about 6 cm can be viewed as acceptable, where a reading of 5.5 cm corresponds to range of about 19,000 centipoises to about 30,000 centipoises at 25 degrees Celsius. It should be understood that various types of Consistometers and Viscometers can be used to test the viscosity of the finished gel. Other characteristics of the gel that can be tested, in some embodiments, include a pH at 20 degrees Celsius expected to lie in a range of about 5.5 to about 6.5, and a specific gravity expected to lie in a range of about 0.990 to about 1.01.

Presuming that the finished gel is found to be acceptable at step 262, then the gel can be transferred to the fragrance containers (e.g., bottles) at step 264, and caps can be screwed onto the filled containers at step 266. Then the containers can be run through a heat sealer at step 268, and labels can be applied to the heat sealed containers at step 270. Finally, a date code can be printed on each container at step 272.

Turning now to FIGS. 30-34 and referring generally thereto, a method of using the water-based fragrance gel 200 with the fragrance warmer 100A is described in detail. With the warmer 100A unplugged, a user can, at step 300, check connection of the 25 watt bulb or replace the 25 watt bulb. Then, at step 302, a user can, with reference to measurement demarcations 350 on the gel container 210, add a predetermined amount (e.g., one-half ounce) of fragrance gel 200 to the warmer tray 104A resting on top of the warmer body 102A. An at least partial light transmissivity of the container 210 can permit the user to visually observe the amount of gel 200 remaining in the container 210 with reference to the indices provided by the measurement demarcations 350 provided on the container 210. If desired, the user can add more or less gel 200, with knowledge that the predetermined amount can be expected to dispense scent to an area for approximately six hours.

At step 304, the user can, with the warmer 100A plugged in, actuate the switch 154 (see FIG. 5) to select one of multiple heat and brightness levels (e.g., high or low) for dispensing scent to an area based on a sized of the area and a desired intensity. Then the user, at step 306, can enjoy the scent for about six hours (depending on the amount dispensed and the heat setting selected) until the gel 200 has observably reduced to a residue 352. Thereafter, at step 308, the user can actuate the switch 154 (see FIG. 5) again to power off the warmer 100A, and allow the warmer tray 104A time to cool. Alternatively or additionally, the user can unplug the warmer in step 308. Once the warmer tray 104A has cooled, the user, at step 310, can remove the residue 352 by hand washing the warmer tray 104A with soap and warm water, and/or by running the warmer tray 104A through a dishwasher. Finally, with the warmer 100A unplugged, the user can, if desired, clean the outside of the warmer body 102A with a soft, damp, clean cloth. It should be understood that this method, although described in particular detail with reference to the first embodiment of the fragrance warmer, can also be employed with other embodiments of the warmer. Moreover, it is envisioned that variations in steps described above, variations in order of steps described above, and/or variations in inclusion of steps described above can be made, while additional steps can also be added.

The foregoing description is of exemplary and preferred embodiments of fragrance warmers, water-based fragrance gels, and related methods of use. The disclosure is not limited to the described examples or embodiments. Alterations and modifications to the disclosed embodiments may be made without departing from the disclosure. The meaning of the terms used in this specification are, unless expressly stated otherwise, intended to have ordinary and customary meaning and are not intended to be limited to the details of the illustrated structures or the disclosed embodiments. None of the foregoing description is to be read as implying that any particular element, step, or function is an essential element which must be included in the claim scope.

The scope of patented subject matter is defined only by the issued claims. None of these claims are intended to invoke paragraph six of 35 USC §112 unless the exact words “means for” or “steps for” are followed by a participle.

What is claimed is:

1. A fragrance warmer, comprising:
a warmer tray;
a warmer body configured to support said warmer tray;
an electrical socket mounted inside said warmer body;
a light bulb electrically interfaced with said electrical socket; and
a switch operatively connected to adjust power supplied to said bulb during activation of said fragrance warmer between at least two heat settings,
wherein said warmer body has heat release features configured to maintain heat of a warming surface of said warmer tray at the at least two heat settings.

2. The fragrance warmer of claim 1, wherein the at least two heat settings greater than zero include a first heat setting of about 135 degrees Fahrenheit, and a second heat setting of about 160 degrees Fahrenheit.

3. The fragrance warmer of claim 1, wherein the heat release features are apertures formed in an upper region of said warmer body.

4. The fragrance warmer of claim 3, wherein the apertures are about ten to about sixteen substantially rounded and/or substantially polygonal openings that are distributed in one or more lines about the upper region of said warmer body, and that have a diameter and/or width residing in a range of about seven millimeters to about twelve millimeters.

5. The fragrance warmer of claim 1, wherein the heat release features are vents formed in an upper lip of said warmer body.

6. The fragrance warmer of claim 1, wherein said warmer body is predominately comprised of at least one of stoneware covered with glaze, unglazed porcelain, or painted polyester resin.

7. The fragrance warmer of claim 1, wherein said light bulb is a 25 Watt, candelabra type, incandescent bulb.

8. The fragrance warmer of claim 1, wherein said electrical socket is mounted vertically to a base of said warmer body by a metallic threaded member extending from a base of said electrical socket through an aperture in the base of said warmer body and supplied with a nut.

9. The fragrance warmer of claim 1, wherein said switch is a single pole throw 2 way switch attached to an electrical cable connected to said electrical socket.

10. The fragrance warmer of claim 1, wherein said warmer tray is predominately comprised of at least one of glazed stoneware, unglazed porcelain, or clear glass having an uneven surface having a light diffusion property.

11. A water-based fragrance gel, comprising:
fragrance;
water;
one or more polymers; and
one or more polyfunctional alcohols.

12. The water-based fragrance gel of claim 11, wherein said water is de-ionized water.

13. The water-based fragrance gel of claim 12, further comprising:
one or more preservatives.

14. The water-based fragrance gel of claim 13, wherein an amount of said fragrance is about 10% by weight, an amount of said de-ionized water resides in a range of about 89.1% to 89.2% by weight, an amount of said one or more polymers resides in a range of about 0.15% to about 0.20% by weight, an amount of said one or more polyfunctional alcohols resides in a range of about 0.15% by weight to about 0.20% by weight, and an amount of said preservative is about 0.5% by weight.

15. The water-based fragrance gel of claim 14, wherein said one or more polymers includes Ultrez 21 polymer.

16. The water-based fragrance gel of claim 14, wherein said one or more polyfunctional alcohols includes triethanolamine.

17. The water-based fragrance gel of claim 14, wherein said one or more preservatives includes phenonip preservative.

18. The water-based fragrance gel of claim 1, wherein said water-based fragrance gel has a viscosity that yields a Consistometer reading residing in a range of about 5 to about 6, and a Consistometer reading of about 5.5 corresponds to a viscosity residing in a range of about 19,000 centipoises to about 30,000 centipoises.

19. A method of making a water-based fragrance gel, the method comprising:
adding water to a clean reactor;
sprinkling one or more polymers into the water without any agitation;
waiting about ten minutes for the polymer to settle down through the water;
blending the polymer and the water at a first agitation speed until a smooth, watery mix is observed;
maintaining the first agitation speed for about two minutes while adding one or more polyfunctional alcohols to the smooth, watery mix, thereby obtaining a thicker mix;
adding a preservative to the thicker mix while maintaining the first agitation speed;
adding a fragrance to the mix and maintaining the first agitation speed until an opaque lotion is observed;
upon observing the opaque lotion, increasing the first agitation speed to a second agitation speed and blending the lotion for about three minutes, thereby obtaining said water-based fragrance gel.

20. The method of claim 19, further comprising:
checking appearance, odor, and viscosity of the water-based fragrance gel, including checking that the viscosity is in or near a range of about 19,000 centipoises to about 30,000 centipoises.

21. The method of claim 19, further comprising:
transferring the water-based fragrance gel to one or more containers that are at least partially light transmissive;
capping the one or more containers;
heat sealing the one or more containers; and
providing one or more demarcations to the one or more containers as indices for dispensing predetermined amounts of the water-based fragrance gel.

22. The method of claim 21, wherein providing the one or more demarcations includes affixing one or more labels to the one or more containers.

23. The method of claim 19, wherein the water is de-ionized water.

24. The method of claim 24, wherein an amount of the fragrance is about 10% by weight, an amount of the de-ionized water resides in a range of about 89.1% to 89.2% by weight, an amount of the one or more polymers resides in a range of about 0.15% to about 0.20% by weight, an amount of the one or more polyfunctional alcohols resides in a range of about 0.15% by weight to about 0.20% by weight, and an amount of the preservative resides in a range of about 0.5% by weight.

25. The water-based fragrance gel of claim 24, wherein the one or more polymers includes Ultrez 21 polymer, the one or more polyfunctional alcohols includes triethanolamine, and the one or more preservatives includes phenonip preservative.

26. A method of using a water-based fragrance gel with a fragrance warmer, the method comprising:
adding an amount of water-based fragrance gel to a warmer tray resting on top of a warmer body of the fragrance warmer;
observing that the water-based gel has reduced to a residue; and
cleaning the residue from the warmer tray, including at least one of hand washing the warmer tray with soap and water, or running the warmer tray through a dishwasher.

27. The method of claim 26, further comprising:
upon observing that the gel has reduced to the residue, deactivating the warmer by at least one of actuating the switch or disconnecting the warmer from a power supply; and
allowing the warmer tray to cool following deactivating the warmer before cleaning the residue from the warmer tray.

28. The method of claim 26, further comprising:
actuating a switch to activate the fragrance warmer by selecting one of at least two heat settings.

29. The method of claim 28, wherein the at least two heat settings include a first heat setting of about 135 degrees Fahrenheit, and a second heat setting of about 160 degrees Fahrenheit.

30. The method of claim 28, wherein actuating the switch activates a 25 Watt, candelabra type, incandescent light bulb in electrical communication with an electrical socket mounted inside the fragrance warmer.

31. The method of claim 28, wherein actuating the switch activates a light bulb in electrical communication with an electrical socket mounted vertically to a base of the warmer body by a metallic threaded member extending from a base of the electrical socket through an aperture in the base of the warmer body and supplied with a nut.

32. The method of claim 28, wherein the switch is a single pole throw 2 way switch attached to an electrical cable connected the fragrance warmer to a power supply.

33. The method of claim 26, further comprising:
with the fragrance warmer disconnected from a power supply, at least one of checking connection of a bulb within the warmer or replacing the bulb.

34. The method of claim 26, further comprising:
with the fragrance warmer disconnected from a power supply, cleaning an exterior the warmer body with a soft, damp, clean cloth.

35. The method of claim 26, wherein the water-based fragrance gel, comprises:
fragrance;
water;
one or more polymers; and
one or more polyfunctional alcohols.

36. The method of claim 35, wherein the water is de-ionized water.

37. The method of claim 36, further comprising:
one or more preservatives.

38. The method of claim 37, wherein an amount of the fragrance is about 10% by weight, an amount of the de-ionized water resides in a range of about 89.1% to 89.2% by weight, an amount of the one or more polymers resides in a range of about 0.15% to about 0.20% by weight, an amount of the one or more polyfunctional alcohols resides in a range of about 0.15% by weight to about 0.20% by weight, and an amount of the preservative is about 0.5% by weight.

39. The method of claim 38, wherein the one or more polymers includes Ultrez 21 polymer, the one or more polyfunctional alcohols includes triethanolamine, and the one or more preservatives includes phenonip preservative.

40. The method of claim 26, wherein the water-based fragrance gel has a viscosity that yields a Consistometer reading residing in a range of about 5 to about 6, and a Consistometer reading of about 5.5 corresponds to a viscosity residing in a range of about 19,000 centipoises to about 30,000 centipoises.
